(19) United States
(12) Patent Application Publication
(10) Pub. No.: US 2016/0074774 A1
(43) Pub. Date: Mar. 17, 2016

(54) CHROMATOGRAPHY METHOD AND SYSTEM

(71) Applicant: Pall Corporation, Port Washington, NY (US)

(72) Inventors: Aleksandar CVETKOVIC, Marlborough, MA (US); Richard A. MARTINO, Brighton, MA (US); Karl K. ROGLER, Lincoln, MA (US)

(21) Appl. No.: 14/483,931

(22) Filed: Sep. 11, 2014

(51) Int. Cl. B01D 15/14; B01D 15/42

(52) U.S. Cl. CPC B01D 15/14; B01D 15/424

(57) ABSTRACT

Chromatography methods and systems are disclosed, wherein a tangential flow filtration device is used to concentrate a product of interest, which improves sorbent utilization.

### BACKGROUND OF THE INVENTION

Chromatography sorbents can be used to purify proteins and polypeptides. For example, chromatography columns containing sorbents can be used in batch mode, wherein the columns are partially loaded due to kinetic limitations of protein/polypeptide adsorption. In some industries, for example, the petrochemical industry, continuous or simulated moving bed chromatography is used to purify a compound of interest, wherein several interconnected columns are simultaneously operated, and the breakthrough of the compound of interest from a loading column is directed to the next column.

However, there is a need for improved chromatography methods and systems. The present invention provides for ameliorating at least some of the disadvantages of the prior art. These and other advantages of the present invention will be apparent from the description as set forth below.

### BRIEF SUMMARY OF THE INVENTION

An embodiment of the invention provides a chromatography system comprising (a) a chromatography device for capturing at least one desired component, the device comprising a housing having an inlet and an outlet and providing a fluid flow path between the inlet and the outlet; and a chromatography medium disposed in the housing across the fluid flow path; (b) a filtration device, comprising a housing comprising an inlet, a first outlet, and a second outlet, defining a first fluid flow path between the inlet and the first outlet, and a second fluid flow path between the inlet and the second outlet, and a porous filter element comprising at least one porous membrane across the first fluid flow path, wherein the second outlet of the filtration device is in fluid communication with the inlet of the chromatography device; (c) an effluent fluid flow path in fluid communication with the outlet of the chromatography device, the effluent fluid flow path comprising at least a first conduit; (d) (di) a sensor associated with the effluent fluid flow path, wherein the sensor detects the presence or absence of the desired component passing from the outlet of the chromatography device along the effluent fluid flow path, and provides a signal indicating of the presence or absence of the desired component, or (dii) a time control arrangement, providing a signal indicating a set period of time with respect to the operation of the chromatography device has elapsed; (e) a first controllable flow control arrangement comprising a first valve, downstream of the outlet of the chromatography device, and downstream of the sensor if present, the first controllable flow control arrangement being in fluid communication with the effluent fluid flow path, wherein, (ei) after the signal indicating of the presence or absence of the desired component is provided, or (eii) after the signal indicating the set period of time has elapsed is provided; the first flow control arrangement allows and/or prevents fluid flow along an eluted component fluid flow path, a first waste fluid flow path, and a desired component recycle fluid flow path; wherein the eluted component fluid flow path, the first waste fluid flow path, and the desired component recycle fluid flow path are downstream of, and in fluid communication with, the first flow control arrangement; (f) the eluted component fluid flow path comprising at least a second conduit; (g) an eluted component container, downstream of, and in fluid communication with, the eluted component fluid flow path; (h) the first waste fluid flow path comprising at least a third conduit; (i) a waste container, downstream of, and in fluid communication with, the first waste fluid flow path; (j) the desired component recycle fluid flow path comprising at least a fourth conduit; (k) a source container, suitable for containing a fluid comprising a desired compoent to be purified; and/or, a mixing vessel, suitable for containing a fluid comprising a desired component to be purified; the source container and/or the mixing vessel in fluid communication with both the desired component recycle fluid flow path and the inlet of the filtration device.

In another embodiment, a chromatography system comprises (a) a chromatography device for capturing at least one desired component, the device comprising a housing having an inlet and an outlet and providing a fluid flow path between the inlet and the outlet; and a chromatography medium disposed in the housing across the fluid flow path; (b) an effluent fluid flow path in fluid communication with the outlet of the chromatography device, the effluent fluid flow path comprising at least a first conduit; (c) (ci) a sensor associated with the effluent fluid flow path, wherein the sensor detects the presence or absence of the desired component passing from the outlet of the chromatography device along the effluent fluid flow path, and provides a signal indicating of the presence or absence of the desired component, or (cii) a time control arrangement, providing a signal indicating a set period of time with respect to the operation of the chromatography device has elapsed; (d) a first controllable flow control arrangement comprising a first valve, downstream of the outlet of the chromatography device, and downstream of the sensor if present, the first controllable flow control arrangement being in fluid communication with the effluent fluid flow path, wherein, after the signal indicative of the presence or absence of the desired component is provided, the flow control arrangement allows and/or prevents fluid flow along an eluted component fluid flow path, a first waste fluid flow path, and a desired component fluid flow path; wherein the eluted component fluid flow path, the first waste fluid flow path, and the desired component fluid flow path are downstream of, and in fluid communication with, the first flow control arrangement; (e) the eluted component fluid flow path comprising at least a second conduit; (f) an eluted component container, downstream of, and in fluid communication with, the eluted component fluid flow path; (g) the first waste fluid flow path comprising at least a third conduit; (h) a waste container, downstream of, and in fluid communication with, the first waste fluid flow path; (i) the desired component fluid flow path comprising at least a fourth conduit; (j) a filtration device, comprising a housing comprising an inlet, a first outlet, and a second outlet, defining a first fluid flow path between the inlet and the first outlet, and a second fluid flow path between the inlet and the second outlet, and a porous filter element comprising at least one porous membrane across the first fluid flow path, wherein the inlet of the filtration device is in fluid communication with the desired component fluid flow path and the second outlet of the filtration device is in fluid communication with a desired component recycle fluid flow path, the desired component recycle fluid flow path comprising at least a fifth conduit; and, (k) a source container, suitable for containing a fluid comprising a desired component to be purified and/or a mixing vessel suitable for containing a fluid comprising a desired component to be purified; the source container and/or the mixing vessel in fluid communication with both the second outlet of the filtration device via the desired component recycle fluid flow path, and the inlet of the chromatography device.

In another embodiment, a chromatographic method for obtaining at least one desired component is provided, the method comprising (a) passing a fluid comprising at least one desired component from a source container or a mixing vessel and into a filtration device, the device comprising a housing comprising an inlet, a first outlet, and a second outlet, defining a first fluid flow path between the inlet and the first outlet, and a second fluid flow path between the inlet and the second outlet, and a porous filter element having an upstream surface and a downstream surface and comprising a porous membrane across the first fluid flow path, wherein the second outlet of the filtration device is in fluid communication with an inlet of a chromatography device; passing the fluid comprising at least one desired component tangentially to the upstream surface of the filter element; passing a desired component concentrated fluid tangentially to the upstream surface and along the second fluid flow path and through the second outlet into the inlet of the chromatography device; and passing a desired component reduced fluid along the first fluid flow path through the filter element and through the first outlet along a second waste fluid flow path to a waste container; (b) passing the desired component concentrated fluid into the chromatography device, the device comprising a housing having the inlet and an outlet and providing a fluid flow path between the inlet and the outlet; and chromatography medium for binding the at least one desired component, disposed in the housing across the fluid flow path; (c) passing fluid from the outlet of the chromatography device along an effluent fluid flow path, including (ci) detecting the presence or absence of the desired component(s) in the fluid, or (cii) determining whether one or more set periods of time have elapsed, the set periods of time comprising a first waste fluid flow time, a desired component recycle fluid flow time, and a eluted component fluid flow path time; (d) opening and/or closing one or more of the following flow paths: a desired component recycle fluid flow path, a first waste fluid flow path, and an eluted component fluid flow path, wherein, (di) if the desired component(s) is not detected, or detected at less than a pre-set value, or (dii) if the desired component recycle fluid flow time has not elapsed, the desired component recycle fluid flow path is opened, and the first waste fluid flow path and the eluted component fluid flow path are closed; and, (diii) if the desired component(s) is detected at equal to, or greater than, the pre-set value, or (div) if the desired component recycle fluid flow time has not elapsed, the desired component recycle fluid flow path is opened or remains open, and the first waste fluid flow path and the eluted component fluid flow path are closed or remain closed; and, (dv) if after the desired component(s) have previously been detected at equal to, or greater than, the pre-set value, and the desired component(s) is subsequently not detected, or detected at less than the pre-set value, or (dvi) if the desired component recycle fluid flow time has elapsed, the desired component recycle fluid flow path and the eluted component fluid flow path are closed, and the waste fluid flow path is opened; (e) passing fluid along the opened fluid flow path, wherein, if the first waste fluid flow path is opened, the method includes passing fluid along the first waste fluid flow path into a waste container, and if the desired component recycle fluid flow path is opened, the method includes passing fluid along the desired component recycle fluid flow path into the source container or into the mixing vessel, and passing fluid from the source container or from the mixing vessel into the tangential fluid flow filter device; and (f) repeating (a), (b), (c), (d) and (e) at least once.

In another embodiment, a chromatographic method for obtaining at least one desired component is provided, the method comprising (a) passing a fluid comprising at least one desired component from a source container or from a mixing vessel and into an inlet of a chromatography device, and through the chromatography device, the device comprising a housing having the inlet and an outlet and providing a fluid flow path between the inlet and the outlet; and a chromatography medium for binding the at least one desired component, disposed in the housing across the fluid flow path; (b) passing fluid from the outlet of the chromatography device along an effluent fluid flow path, including (bi) detecting the presence or absence of the desired component(s) in the fluid, or (bii) determining whether one or more set periods of time have elapsed, the set periods of time comprising a first waste fluid flow time, a desired component fluid flow time, and an eluted component fluid flow path time; (c) opening and/or closing one or more of the following flow paths: a desired component fluid flow path, a first waste fluid flow path, and an eluted component fluid flow path, wherein, (ci) if the desired component(s) is not detected, or detected at less than a pre-set value, or (cii) if the desired component fluid flow time has not elapsed, the desired component fluid flow path is opened, and the first waste fluid flow path and the eluted component fluid flow path are closed; and, (ciii) if the desired component(s) is detected at equal to, or greater than, the pre-set value, or (civ) if the desired component fluid flow time has not elapsed, the desired component fluid flow path is opened or remains open, and the first waste fluid flow path and the eluted component fluid flow path are closed or remain closed; and, (cv) if after the desired component(s) have previously been detected at equal to, or greater than, the pre-set value, and the desired component(s) is subsequently not detected, or detected at less than the pre-set value, or (cvi) if the desired component fluid flow time has elapsed, the desired component fluid flow path and the eluted component fluid flow path are closed, and the waste fluid flow path is opened; (d) passing fluid along the opened fluid flow path, wherein, if the first waste fluid flow path is opened, the method includes passing fluid along the first waste fluid flow path into a waste container, and if the desired component fluid flow path is opened, the method includes passing fluid along the desired component recycle fluid flow path into an inlet of a tangential fluid flow filter device, the tangential flow filtration device comprising a housing comprising the inlet, a first outlet, and a second outlet, defining a first fluid flow path between the inlet and the first outlet, and a second fluid flow path between the inlet and the second outlet, and a porous filter element having an upstream surface and a downstream surface and comprising a porous membrane across the first fluid flow path, wherein the second outlet of the tangential flow filtration device is in fluid communication with the source container and/or with the mixing vessel via a desired component recycle fluid flow path, wherein the source container and/or the mixing vessel is in fluid communication with both the second outlet of the tangential flow filtration device via the desired component recycle fluid flow path, and the inlet of the chromatography device; (e) passing the fluid comprising at least one desired component tangentially to the upstream surface of the filter element; passing a desired component concentrated fluid tangentially to the upstream surface and along the second fluid flow path and through the second outlet along the desired component recycle fluid flow path into the source container or into the mixing vessel; and passing a desired component reduced fluid along the first fluid flow path through the filter element and through the first outlet along a second waste fluid flow path to the waste container; and (f) repeating (a), (b), (c), (d) and (e) at least once.

Typically, embodiments of the chromatographic method for obtaining at least one desired component further comprise (g) closing the desired component recycle flow path or the desired component flow path, and the first waste fluid flow path, and opening the eluted component fluid flow path; and; (h) passing an elution fluid through the chromatography device, and passing desired component(s) containing elution fluid from the outlet of the chromatograph device along the eluted component fluid flow path into the eluted component container.

### BRIEF DESCRIPTION OF THE SEVERAL VIEWS OF THE DRAWING(S)

FIG. 1 shows, diagrammatically, an embodiment of a chromatography system according to the present invention, showing a tangential flow filtration (TFF) device arranged in a fluid flow path between a source container (one option) or a mixing vessel (another option), and a chromatography device, wherein the TFF device is upstream of the chromatography device, and a product detector (sensor) is associated with an effluent fluid flow path downstream of the chromatography device. FIG. 1 also shows an optional surge tank, downstream of the TFF device, and upstream of the chromatography device.

FIG. 2 shows, diagrammatically, another embodiment of a chromatography system according to the present invention, showing a TFF device arranged in a fluid flow path between a chromatography device and a source container (one option) or a mixing vessel (another option), wherein the TFF device is downstream of the chromatography device, and a product detector (sensor) is associated with an effluent fluid flow path downstream of the chromatography device. FIG. 2 also shows an optional surge tank, downstream of the TFF device, and upstream of the chromatography device.

FIG. 3 shows, diagrammatically, an embodiment of a chromatography system according to the present invention, showing a TFF device arranged in a fluid flow path between a source container (one option) or a mixing vessel (another option), and a chromatography device, wherein the TFF device is upstream of the chromatography device, and the system also includes a time control arrangement. FIG. 3 also shows an optional surge tank, upstream of the TFF device, and downstream of the chromatography device.

FIG. 4 shows, diagrammatically, another embodiment of a chromatography system according to the present invention, showing a TFF device arranged in a fluid flow path between a chromatography device and a source container (one option) or a mixing vessel (another option), wherein the TFF device is downstream of the chromatography device, and the system also includes a time control arrangement. FIG. 4 also shows an optional surge tank, upstream of the TFF device, and downstream of the chromatography device.

### DETAILED DESCRIPTION OF THE INVENTION

A filtration device (such as a tangential fluid flow filter device) is used to improve the concentration and/or purification of one or more desired components in a fluid, and in accordance with an embodiment of the present invention, usability of the medium or media in the chromatography device is improved when the filtration device is used with: a chromatography device; (i) a sensor associated with the effluent fluid flow path of the chromatography device, the sensor detecting the presence or absence of at least one desired component passing from the outlet of the chromatography device, and producing a signal indicative of the presence of absence of the desired component(s), or (ii) a time control arrangement, providing a signal indicating a set period of time with respect to the operation of the chromatography device has elapsed; and a controllable flow control arrangement comprising a valve, downstream of the outlet of the chromatography device, and downstream of the sensor if present, the controllable flow control arrangement being in fluid communication with the effluent fluid flow path, wherein, (i) after the signal indicative of the presence or absence of the desired component(s) is produced, or (ii) after the signal indicating the set period of time has elapsed is provided; the flow control arrangement allows and/or prevents fluid flow along an eluted component fluid flow path, a waste fluid flow path, and a desired component recycle fluid flow path or a desired component fluid flow path; wherein the eluted component fluid flow path, the waste fluid flow path, and the desired component recycle fluid flow path and the desired component fluid flow path are downstream of, and in fluid communication with, the flow control arrangement. In accordance with embodiments of the invention, the filtration device can be arranged, for example, after the chromatography device and before a source container and/or a mixing vessel and/or between the source container or the mixing vessel and the chromatography device.

Advantageously, in contrast with conventional systems, wherein media in chromatography devices used in batch mode are only partially loaded due to kinetic limitations of protein adsorption (dynamic binding capacities (DBCs)) are commonly between about 30-50% of their static binding capacities), media can be used more efficiently in accordance with the invention. Other advantages include an increase in specific productivity and improved process economics (e.g., reduced resin volume, reduced buffer and water usage, and faster processing).

An embodiment of the invention provides a chromatography system comprising (a) a chromatography device for capturing at least one desired component, the device comprising a housing having an inlet and an outlet and providing a fluid flow path between the inlet and the outlet; and a chromatography medium disposed in the housing across the fluid flow path; (b) a tangential flow fluid filtration device, comprising a housing comprising an inlet, a first outlet, and a second outlet, defining a first fluid flow path between the inlet and the first outlet, and a second fluid flow path between the inlet and the second outlet, and a porous filter element comprising at least one porous membrane across the first fluid flow path, wherein the second outlet of the filtration device is in fluid communication with the inlet of the chromatography device; (c) an effluent fluid flow path in fluid communication with the outlet of the chromatography device, the effluent fluid flow path comprising at least a first conduit; (d) (di) a sensor associated with the effluent fluid flow path, wherein the sensor detects the presence or absence of the desired component(s) passing from the outlet of the chromatography device along the effluent fluid flow path, and provides a signal indicating of the presence or absence of the desired component(s), or (dii) a time control arrangement, providing a signal indicating a set period of time with respect to the operation of the chromatography device has elapsed; (e) a first controllable flow control arrangement comprising a first valve, downstream of the outlet of the chromatography device, and downstream of the sensor if present, the first controllable flow control arrangement being in fluid communication with the effluent fluid flow path, wherein, (ei) after the signal indicating of the presence or absence of the desired component(s) is provided, or (eii) after the signal indicating the set period of time has elapsed is provided; the first flow control arrangement allows and/or prevents fluid flow along an eluted component fluid flow path, a first waste fluid flow path, and a desired component recycle fluid flow path; wherein the eluted component fluid flow path, the first waste fluid flow path, and the desired component recycle fluid flow path are downstream of, and in fluid communication with, the first flow control arrangement; (f) the eluted component fluid flow path comprising at least a second conduit; (g) an eluted component container, downstream of, and in fluid communication with, the eluted component fluid flow path; (h) the first waste fluid flow path comprising at least a third conduit; (i) a waste container, downstream of, and in fluid communication with, the first waste fluid flow path; (j) the desired component recycle fluid flow path comprising at least a fourth conduit; (k) a source container, suitable for containing a fluid comprising a desired component to be purified and/or a mixing vessel, suitable for containing a fluid comprising a desired component to be purified; the source container and/or the mixing vessel in fluid communication with both the desired component recycle fluid flow path and the inlet of the filtration device.

Alternatively, or additionally, an embodiment of the system further comprises a surge tank, downstream of the tangential flow filtration device and in fluid communication with the second outlet of the filtration device, and upstream of the inlet of the inlet of the chromatography device. In a preferred embodiment, the system further comprises a second controllable flow control arrangement comprising a second valve, interposed between, and in fluid communication with, the second outlet of the tangential flow filtration device and the inlet of the chromatography device, wherein the additional controllable flow control arrangement allows and/or prevents fluid flow from the second outlet to the inlet of the chromatography device, and from one or more buffer containers to the inlet of the chromatography device.

In another embodiment, a chromatography system comprises (a) a chromatography device for capturing at least one desired component, the device comprising a housing having an inlet and an outlet and providing a fluid flow path between the inlet and the outlet; and a chromatography medium disposed in the housing across the fluid flow path; (b) an effluent fluid flow path in fluid communication with the outlet of the chromatography device, the effluent fluid flow path comprising at least a first conduit; (c) (ci) a sensor associated with the effluent fluid flow path, wherein the sensor detects the presence or absence of the desired component passing from the outlet of the chromatography device along the effluent fluid flow path, and provides a signal indicating of the presence or absence of the desired component, or (cii) a time control arrangement, providing a signal indicating a set period of time with respect to the operation of the chromatography device has elapsed; (d) a first controllable flow control arrangement comprising a first valve, downstream of the outlet of the chromatography device, and downstream of the sensor if present, the first controllable flow control arrangement being in fluid communication with the effluent fluid flow path, wherein, after the signal indicative of the presence or absence of the desired component is provided, the flow control arrangement allows and/or prevents fluid flow along an eluted component fluid flow path, a first waste fluid flow path, and a desired component fluid flow path; wherein the eluted component fluid flow path, the first waste fluid flow path, and the desired component fluid flow path are downstream of, and in fluid communication with, the first flow control arrangement; (e) the eluted component fluid flow path comprising at least a second conduit; (f) an eluted component container, downstream of, and in fluid communication with, the eluted component fluid flow path; (g) the first waste fluid flow path comprising at least a third conduit; (h) a waste container, downstream of, and in fluid communication with, the first waste fluid flow path; (i) the desired component fluid flow path comprising at least a fourth conduit; (j) a filtration device, comprising a housing comprising an inlet, a first outlet, and a second outlet, defining a first fluid flow path between the inlet and the first outlet, and a second fluid flow path between the inlet and the second outlet, and a porous filter element comprising at least one porous membrane across the first fluid flow path, wherein the inlet of the filtration device is in fluid communication with the desired component fluid flow path, and the second outlet of the filtration device is in fluid communication with a desired component recycle fluid flow path, the desired component recycle fluid flow path comprising at least a fifth conduit; and, (k) a source container, suitable for containing a fluid comprising a desired component to be purified and/or a mixing vessel, suitable for containing a fluid comprising a desired component to be purified; the source container and/or the mixing vessel in fluid communication with both the second outlet of the filtration device via the desired component recycle fluid flow path, and the inlet of the chromatography device.

Alternatively, or additionally, an embodiment of the system further comprises a surge tank, downstream of the outlet of the chromatography device and in fluid communication with the effluent fluid flow path, and upstream of the tangential flow filtration device and in fluid communication with the inlet of the filtration device. In a preferred embodiment, the system further comprises a second controllable flow control arrangement comprising a second valve, interposed between, and in fluid communication with, the source container and/or the mixing vessel, and the inlet of the chromatography device, wherein the second controllable flow control arrangement allows and/or prevents fluid flow from the source container and/or the mixing vessel and from one or more buffer containers to the inlet of the chromatography device.

Typically, embodiments of the systems further comprise a second waste fluid flow path, comprising an additional conduit, in fluid communication with the first outlet of the tangential flow filtration device and the waste container.

In some embodiments of the system, the sensor detects the presence of the desired component compared to a pre-set value.

In some embodiments of the system, the chromatography device comprises at least one chromatography column, at least one chromatography monolith, at least one chromatography capsule, or at least one chromatography cartridge. However, embodiments of the invention can comprise a plurality of chromatography devices, or the same type, or combinations of different types.

In another embodiment, a chromatographic method for obtaining at least one desired component is provided, the method comprising (a) passing a fluid comprising at least one desired component from a source container and/or from a mixing vessel and into a filtration device, the device comprising a housing comprising an inlet, a first outlet, and a second outlet, defining a first fluid flow path between the inlet and the first outlet, and a second fluid flow path between the inlet and the second outlet, and a porous filter element having an upstream surface and a downstream surface and comprising a porous membrane across the first fluid flow path, wherein the second outlet of the filtration device is in fluid communication with an inlet of a chromatography device; passing the fluid comprising at least one desired component tangentially to the upstream surface of the filter element; passing a desired component concentrated fluid tangentially to the upstream surface and along the second fluid flow path and through the second outlet into the inlet of the chromatography device; and passing a desired component reduced fluid along the first fluid flow path through the filter element and through the first outlet along a second waste fluid flow path to a waste container; (b) passing the desired component concentrated fluid into the chromatography device, the device comprising a housing having the inlet and an outlet and providing a fluid flow path between the inlet and the outlet; and chromatography medium for binding the at least one desired component, disposed in the housing across the fluid flow path; (c) passing fluid from the outlet of the chromatography device along an effluent fluid flow path, including (ci) detecting the presence or absence of the desired component(s) in the fluid, or (cii) determining whether one or more set periods of time have elapsed, the set periods of time comprising a first waste fluid flow time, a desired component recycle fluid flow time, and a eluted component fluid flow path time; (d) opening and/or closing one or more of the following flow paths: a desired component recycle fluid flow path, a first waste fluid flow path, and an eluted component fluid flow path, wherein, (di) if the desired component(s) is not detected, or detected at less than a pre-set value, or (dii) if the desired component recycle fluid flow time has not elapsed, the desired component recycle fluid flow path is opened, and the first waste fluid flow path and the eluted component fluid flow path are closed; and, (diii) if the desired component(s) is detected at equal to, or greater than, the pre-set value, or (div) if the desired component recycle fluid flow time has not elapsed, the desired component recycle fluid flow path is opened or remains open, and the first waste fluid flow path and the eluted component fluid flow path are closed or remain closed; and, (dv) if after the desired component(s) have previously been detected at equal to, or greater than, the pre-set value, and the desired component(s) is subsequently not detected, or detected at less than the pre-set value, or (dvi) if the desired component recycle fluid flow time has elapsed, the desired component recycle fluid flow path and the eluted component fluid flow path are closed, and the waste fluid flow path is opened; (e) passing fluid along the opened fluid flow path, wherein, if the first waste fluid flow path is opened, the method includes passing fluid along the first waste fluid flow path into a waste container, and if the desired component recycle fluid flow path is opened, the method includes passing fluid along the desired component recycle fluid flow path into the source container or into the mixing vessel, and passing fluid from the source container or from the mixing vessel into the tangential fluid flow filter device; and (f) repeating (a), (b), (c), (d) and (e) at least once.

In another embodiment, a chromatographic method for obtaining at least one desired component is provided, the method comprising (a) passing a fluid comprising at least one desired component from a source container and/or from a mixing vessel and into an inlet of a chromatography device, and through the chromatography device, the device comprising a housing having the inlet and an outlet and providing a fluid flow path between the inlet and the outlet; and a chromatography medium for binding the at least one desired component, disposed in the housing across the fluid flow path; (b) passing fluid from the outlet of the chromatography device along an effluent fluid flow path, including (bi) detecting the presence or absence of the desired component(s) in the fluid, or (bii) determining whether one or more set periods of time have elapsed, the set periods of time comprising a first waste fluid flow time, a desired component fluid flow time, and a eluted component fluid flow path time; (c) opening and/or closing one or more of the following flow paths: a desired component fluid flow path, a first waste fluid flow path, and an eluted component fluid flow path, wherein, (ci) if the desired component(s) is not detected, or detected at less than a pre-set value, or (cii) if the desired component fluid flow time has not elapsed, the desired component fluid flow path is opened, and the first waste fluid flow path and the eluted component fluid flow path are closed; and, (ciii) if the desired component(s) is detected at equal to, or greater than, the pre-set value, or (civ) if the desired component fluid flow time has not elapsed, the desired component fluid flow path is opened or remains open, and the first waste fluid flow path and the eluted component fluid flow path are closed or remain closed; and, (cv) if after the desired component(s) have previously been detected at equal to, or greater than, the pre-set value, and the desired component(s) is subsequently not detected, or detected at less than the pre-set value, or (cvi) if the desired component fluid flow time has elapsed, the desired component fluid flow path and the eluted component fluid flow path are closed, and the waste fluid flow path is opened; (d) passing fluid along the opened fluid flow path, wherein, if the first waste fluid flow path is opened, the method includes passing fluid along the first waste fluid flow path into a waste container, and if the desired component fluid flow path is opened, the method includes passing fluid along the desired component fluid flow path into an inlet of a tangential fluid flow filter device, the tangential flow filtration device comprising a housing comprising the inlet, a first outlet, and a second outlet, defining a first fluid flow path between the inlet and the first outlet, and a second fluid flow path between the inlet and the second outlet, and a porous filter element having an upstream surface and a downstream surface and comprising a porous membrane across the first fluid flow path, wherein the second outlet of the tangential flow filtration device is in fluid communication with the source container and/or with the mixing vessel via a desired component recycle fluid flow path, wherein the source container or the mixing vessel is in fluid communication with both the second outlet of the tangential flow filtration device via the desired component recycle fluid flow path, and the inlet of the chromatography device; (e) passing the fluid comprising at least one desired component tangentially to the upstream surface of the filter element; passing a desired component concentrated fluid tangentially to the upstream surface and along the second fluid flow path and through the second outlet along the desired component recycle fluid flow path into the source container or into the mixing vessel; and passing a desired component reduced fluid along the first fluid flow path through the filter element and through the first outlet along a second waste fluid flow path to the waste container; and (f) repeating (a), (b), (c), (d) and (e) at least once.

If desired, some embodiments of the method, particularly some embodiments involving the use of a time control arrangement rather than a sensor, further comprise setting the volumetric concentration factor (VCF) to keep the concentration of the desired (target) component stable at the inlet of the chromatography column. Flow rates at the inlet and permeate outlet are set to maintain the mass balance of target component in the chromatography system such that mass added via the source container or the mixing vessel equals the mass removed via the chromatography device.

As noted above, in some embodiments, fluid is passed from or into the source container, and in other embodiments, fluid is passed from or into a mixing vessel. For example, embodiments of the method can include passing fluid along the desired component recycle fluid flow path into the source container, and passing fluid from the source container into the TFF device, or passing fluid along the desired component recycle fluid flow path into the mixing vessel, and passing fluid from the mixing vessel into the TFF device; or passing fluid along the desired component fluid flow path into an inlet of a TFF device, the TFF device comprising a housing comprising the inlet, a first outlet, and a second outlet, defining a first fluid flow path between the inlet and the first outlet, and a second fluid flow path between the inlet and the second outlet, and a porous filter element having an upstream surface and a downstream surface and comprising a porous membrane across the first fluid flow path, wherein the second outlet of the TFF device is in fluid communication with the source container; or passing fluid along the desired component fluid flow path into an inlet of a TFF device, the TFF device comprising a housing comprising the inlet, a first outlet, and a second outlet, defining a first fluid flow path between the inlet and the first outlet, and a second fluid flow path between the inlet and the second outlet, and a porous filter element having an upstream surface and a downstream surface and comprising a porous membrane across the first fluid flow path, wherein the second outlet of the tangential flow filtration device is in fluid communication with the mixing vessel. In some embodiments including the use of a mixing vessel, fluid is initially passed from the source container and into the mixing vessel during the sample load phase and thereafter, fluid is passed into and from the mixing vessel, without passing fluid into and from source container.

Alternatively, or additionally, in some embodiments, a surge tank is interposed between, and in fluid communication with, the chromatography device and the TFF device, such that embodiments of the method can comprise passing fluid from a TFF device into a surge tank and subsequently into the inlet of a chromatography device, or comprise passing fluid along an effluent fluid flow path into a surge tank and subsequently into a tangential flow filtration device.

Typically, embodiments of the chromatographic method for obtaining at least one desired component further comprise (g) closing the desired component recycle flow path or the desired component flow path, and the first waste fluid flow path, and opening the eluted component fluid flow path; and; (h) passing an elution fluid through the chromatography device, and passing the desired component(s) containing elution fluid from the outlet of the chromatograph device along the eluted component fluid flow path into the eluted component container.

Typically, after passing the desired component(s) containing elution fluid from the outlet of the chromatograph device along the eluted component fluid flow path into the eluted component container, the method further comprises passing a cleaning and/or sanitation buffer, and/or an equilibrium buffer (e.g., from a cleaning and/or sanitation buffer container and/or from an equilibrium buffer container) through the chromatography device and into the waste container, to complete a chromatography cycle. Cycles can be repeated as described above.

Each of the components of the invention will now be described in more detail below, wherein like components have like reference numbers.

A wide variety of chromatography devices including chromatography media are suitable for carrying out the invention, for example, devices including beads (e.g., chromatography columns), or including one or more porous chromatography membranes, or including one or more chromatography monoliths (e.g., polymerized gels, silica columns, ceramics, graphitized carbon). A chromatography device can include any number of, e.g., beads, chromatography membranes or chromatography monoliths. A chromatography device can include a plurality (i.e., two or more) of the same type of media, or different types of media. Alternatively, or additionally, a chromatography device can include a plurality of the same type of media wherein the media have different characteristics, e.g., chromatography types. Suitable chromatography devices are known in the art.

In some embodiments, the chromatography device, e.g., comprising a housing and the chromatography medium or chromatography media sealed therein, is a preassembled device, e.g., wherein the medium/media is/are sealed in the housing by the device manufacturer. In some other embodiments, the medium/media is/are sealed in the housing by the end user. The devices can be suitable for treating a variety of fluids, e.g., to purify and/or concentrate one or more desired materials present in the fluids. For example, the devices can be suitable for treating process fluids such as fluids used in the biopharmaceutical industry, e.g., fluids including desirable material such as proteinaceous material, for example, antibodies (e.g., monoclonal antibodies), or recombinant proteins such as growth factors.

Chromatography devices (e.g., capsules, cartridges, and columns), the housings, membranes, and/or monoliths can have any suitable configuration, including, but not limited to, configurations known in the art. For example, the membranes can be one or more of the following forms: planar, pleated, hollow cylindrical, hollow fiber, stacked, and spiral wound. Illustratively, in one embodiment, the membrane(s) can be in the form of a hollow, generally cylindrical, pleated element. The monoliths can be one or more of the following forms: disk, tube, and column.

Additionally, embodiments of the invention encompass the use of a plurality of chromatography devices, e.g., arranged in series and/or in parallel.

Thus, for example, fluid comprising at least one desired component can be passed into the inlet of a first chromatography device and through the outlet to the first device and into the inlet of a second chromatography device, and through the outlet of the second device and along an effluent fluid flow path and through a controllable flow control arrangement that opens and/or closes fluid flow paths as described herein. There can be more than two chromatography devices arranged in series. Alternatively, for example, fluid can be passed into the inlets of two or more chromatography devices (e.g., essentially simultaneously) and passed through the outlets of the devices, and combined and passed along an effluent fluid flow path and through a controllable flow control arrangement that opens and/or closes fluid flow paths as described herein.

As noted above, embodiments can include a plurality of types of chromatography devices, e.g., at least one chromatography column; and, at least one chromatography monolith and/or at least one chromatography capsule.

The substrate media, e.g., beads, membrane(s), monolith(s), can have any desired characteristics, e.g., chromatography type, and a variety of beads, membranes and monoliths (and chromatography devices such as chromatography capsules and chromatography cartridges), including commercially available beads, membranes and monoliths (and commercially available chromatography devices containing such membranes or monoliths), can be used in accordance with the invention, and are known in the art. For example, a variety of ion-exchange beads, membranes and/or monoliths, including a variety of commercially available ion-exchange beads, membranes and/or monoliths, can be used. In some embodiments, the media can function under physiological pH and/or ionic strength. Suitable beads (e.g., resins), membranes and/or monoliths include charged (e.g., a positively charged or a negatively charged media), mixed-mode chromatography media, hydrophobic interactive chromatography (HIC) media, affinity chromatography (e.g., immobilized metal affinity chromatography (IMAC)) media, biospecific (e.g., immobilized Protein A) affinity chromatography media, hydrophobic charge induction chromatography (HCIC) media, and a thiophilic chromatography (TC) media.

A variety of filtration devices comprising a housing comprising at least one inlet and at least one outlet, and defining a fluid flow path between the inlet and the outlet, and at least one porous membrane across the fluid flow path, are suitable and are known in the art. Preferably, the filtration device comprises a TFF device (in some embodiments, a single pass TFF (SP TTF) device), and a variety of TFF devices are suitable and are known in the art. Typically, a TFF device, sometimes referred to as a crossflow filter module, comprises a housing comprising at least one inlet and at least a first outlet (e.g., “a permeate outlet”) and a second outlet (e.g., “a concentrate outlet” or a “retentate outlet”) and defining at least a first fluid flow path between the inlet and the first outlet, and a second fluid flow path between the inlet and the second outlet, the device further comprising at least one porous filter element comprising one or more porous membrane(s) (including hollow fiber membranes) across the first fluid flow path. As will be described in more detail below, in accordance with embodiments of the invention, the TFF device can be operated upstream of, and/or downstream of, a chromatography device.

A variety of sensors are suitable and are known in the art. Suitable sensors include, for example, spectrometer flow cells and detectors used in spectroscopy, such as UV, infra-red, NIR, X-ray, visible, fluorescence, NMR, and Raman detectors.

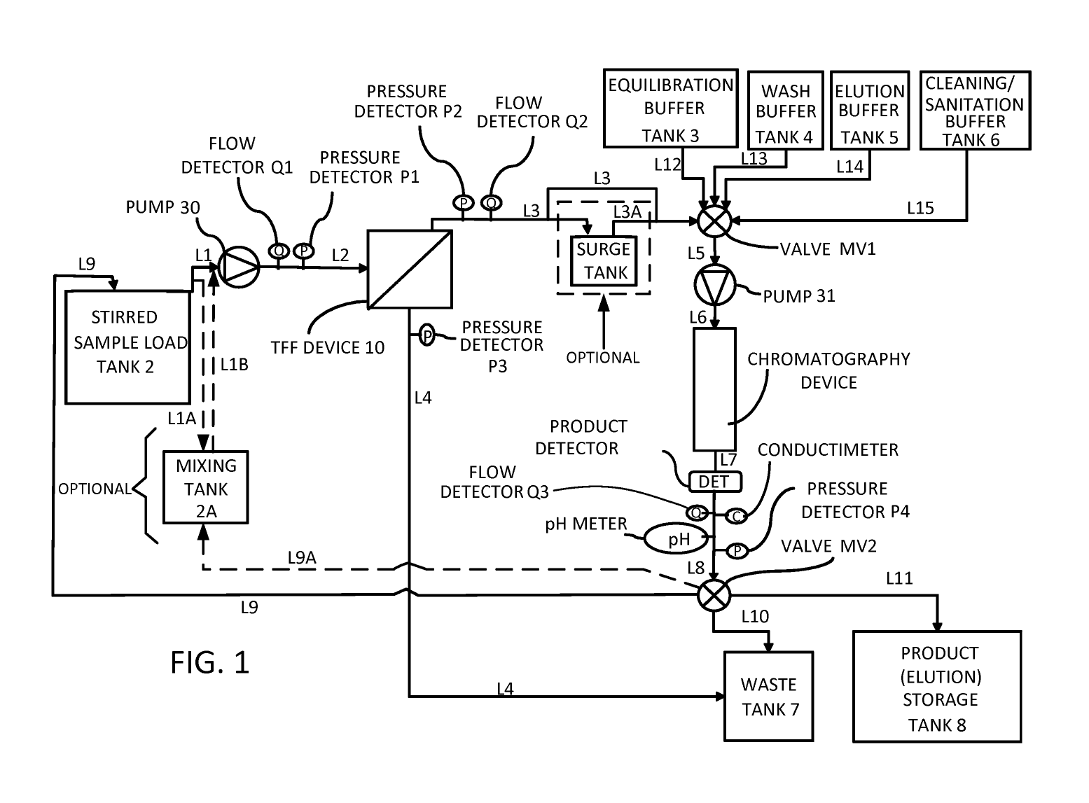

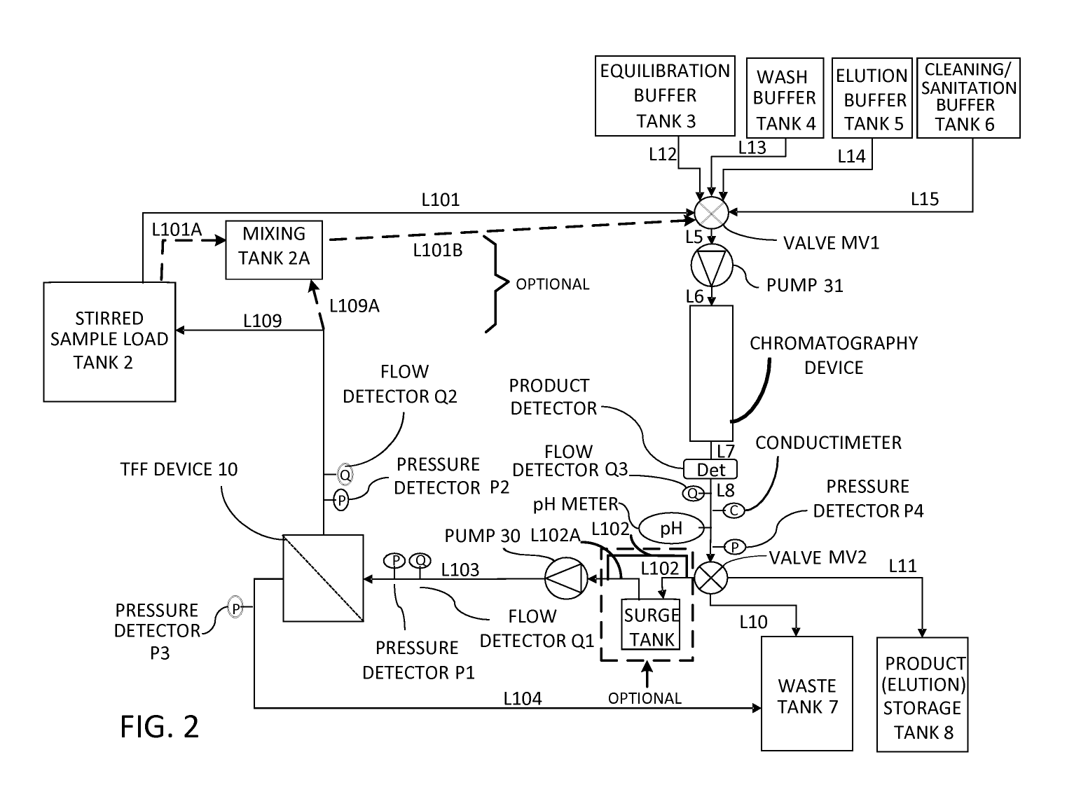

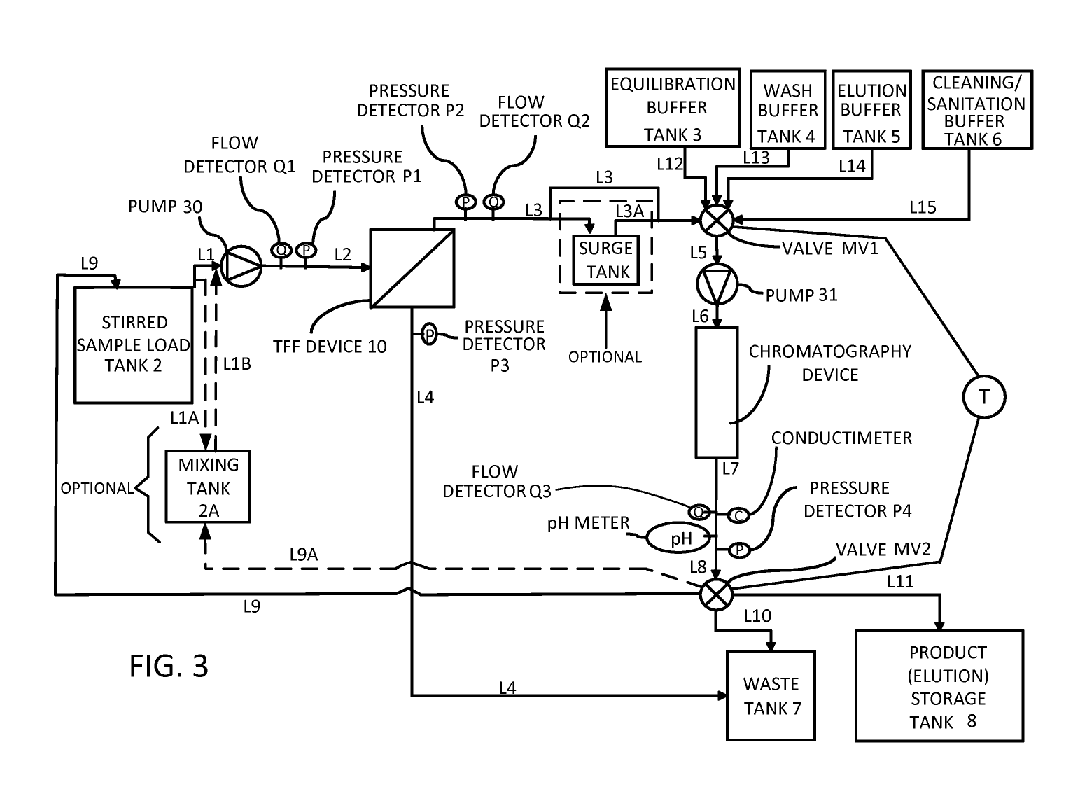

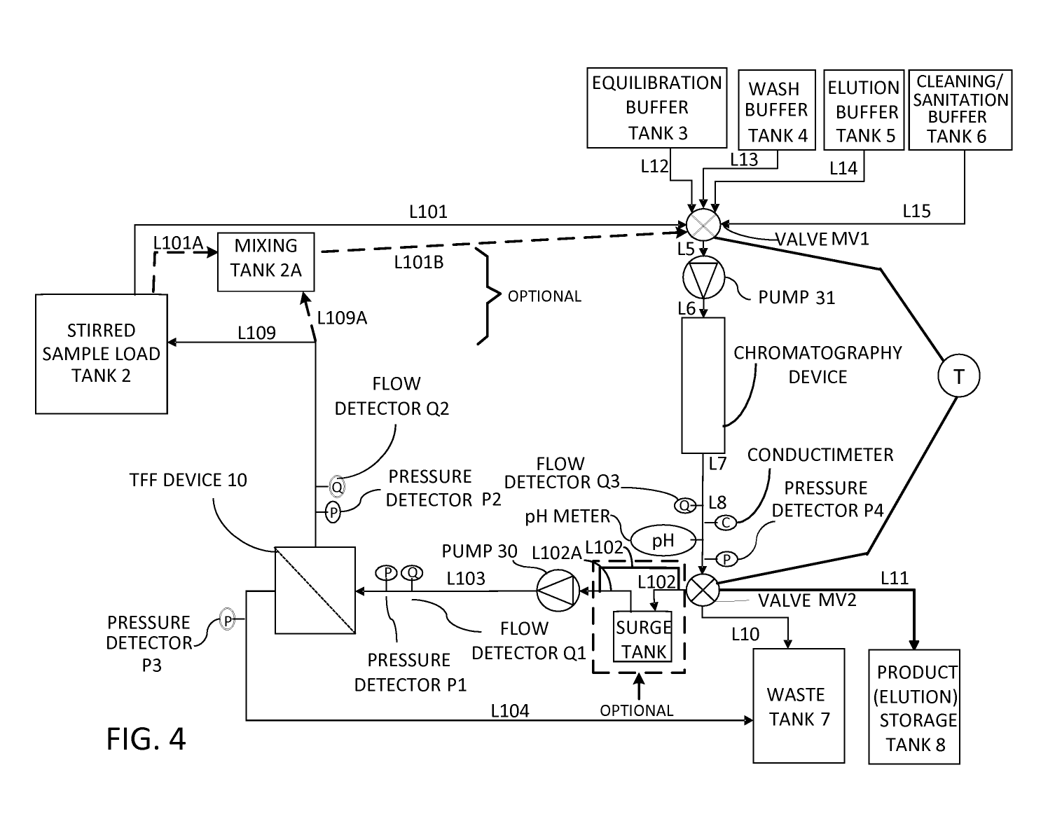

Embodiments of the system typically include one or more pumps, such as variable speed pumps. Preferably, as illustrated in the Figures, the systems include at least two pumps (30, 31), illustrated in FIGS. 1 and 3 as interposed between the TFF device (10) and the source container (2; illustrated as “stirred sample load tank”) and mixing tank (2A), and between a fluid flow control arrangement (MV1) and the inlet of a chromatography device, and illustrated in FIGS. 2 and 4 as interposed between a fluid flow control arrangement (MV1) and the inlet of a chromatography device, and between another flow control arrangement (MV2) and the inlet of the TFF device (10). Suitable pumps are known in the art.

A variety of controllable fluid flow arrangements comprising valves (including manually controllable and software controllable flow arrangements) are suitable, and are known in the art. Typically, as illustrated in the Figures, the systems include at least two controllable fluid flow arrangements upstream (MV1) and downstream (MV2) of the chromatography devices.

A variety of time control arrangements are suitable, and are known in the art. For example, a time control arrangement can comprise software monitoring the time and/or a time control arrangement can comprise a device such as a timer. While the FIGS. 2 and 4 illustrate time control arrangement “T” in relation to upstream and downstream flow control arrangements MV1 and MV2, this is merely a diagrammatic illustration encompassing software and/or a specific device.

If desired, the system can include one or more of any of the following: a pressure detector (e.g., a pressure gauge), a flow detector, a conductivity detector, and a pH detector, as is/are known in the art. While not required, the exemplary systems illustrated in the Figures include a plurality of pressure detectors (“P,” shown as P1, P2, P3, and P4) and/or pH detectors (“pH”), a plurality of flow detectors (“Q,” shown as flow detector Q1, Q2, and Q3), and a conductivity detector (“C,” shown as “conductimeter”).

The chromatography device can be of any suitable dimension and volume. With respect to chromatography columns and/or monoliths, for example, separations on a laboratory scale may warrant a column volume as small as, for example, about 1 milliliter or even about 1 microliter. Large scale purification and isolation of desired components can be performed on columns and/or monoliths as large as, for example, about 5000 liters. More typical volumes are, for example, between 1 liter and 100 liters.

The systems and methods of the present invention can be used to separate and, if desired, purify, a variety of desired components, including biologically relevant molecules and biological substances such as antibodies, proteins, glycoproteins, fusion proteins, recombinant proteins, tagged proteins, enzymes and biological catalysts, peptides, cells, bacteria, viruses, virus-like particles (VLPs), vaccines, nucleic acids, carbohydrates, and lipids. Other substances that are suitable for separation (and, if desired, purification) include oligo- and polysaccharides, lipopolysaccharides, polypeptides, and synthetic soluble polymers. The desired components (e.g., biological substances) typically derive from, or are contained in, sources including but not limited to liquid samples such as saliva, biological fluid, urine, lymphatic fluid, prostatic fluid, seminal fluid, milk, milk whey, organ extracts, plant extracts, cell extracts, cell cultures (including cell lines), fermentation broths, serum, ascites fluid, and transgenic plant and animal extracts. As used herein, a biological fluid includes any treated or untreated fluid associated with living organisms, particularly blood, including whole blood, warm or cold blood, cord blood, and stored or fresh blood; treated blood, such as blood diluted with at least one physiological solution, including but not limited to saline, nutrient, and/or anticoagulant solutions; blood components, such as platelet concentrate (PC), platelet-rich plasma (PRP), platelet-poor plasma (PPP), platelet-free plasma, plasma, fresh frozen plasma (FFP), components obtained from plasma, packed red cells (PRC), transition zone material or buffy coat (BC); blood products derived from blood or a blood component or derived from bone marrow; leukocytes, stem cells; red cells separated from plasma and resuspended in a physiological solution or a cryoprotective fluid; and platelets separated from plasma and resuspended in a physiological solution or a cryoprotective fluid. A biological fluid also includes a physiological solution comprising a bone marrow aspirate. The biological fluid may have been treated to remove some of the leukocytes before being used according to the invention. Blood product or biological fluid refers to the components described above, and to similar blood products or biological fluids obtained by other means and with similar properties.

In this context, one preferred class of biological substances is immunoglobulins. The “immunoglobulins” category embraces whole immunoglobulins, including monoclonal and polyclonal antibodies, as well as Fab, F(ab′)₂, F_{c} and Fᵥ fragments, and other engineered antibody species. In an embodiment, the immunoglobulin may be immunoglobulin G (IgG). Alternatively, or additionally, one or more of any of the following: IgA, IgM, IgD and IgE, can be bound. In some embodiments, IgA and/or IgM are selectively bound.

In accordance with embodiments of the invention, the systems (e.g., the chromatography device and TFF device) are equilibrated with a buffer before carrying out embodiments of the method. Suitable equilibration buffers and methods are known in the art.

Typically, in carrying out embodiments of the method, the contents of the source container are mixed as appropriate, and fluid is passed to the chromatography device at a predetermined flow rate, and the fluid (feed) contacts the chromatography medium for a period of time sufficient to allow at least one desired component (e.g., at least one desired biological substance) to bind to the medium. Typically, the contact period is between about 30 seconds to about 12 hours.

If appropriate, the pH, ionic strength, or both, of the liquid may be adjusted prior to placing the liquid in contact with the chromatography medium. Additionally, or alternatively, the liquid may be concentrated, diluted, or mixed with additives such as salts. Typical capture pH values for a range of proteins is from about 4 to about 10, although the capture pH can be higher or lower. Typically, a pH in the range of about 4 to about 8 promotes protein adsorption to those substrates that include a cation exchange moiety, while a pH in the range of about 6 to about 10 will accomplish the same where anion exchange moieties are used. In some embodiments, the medium may bind proteins at a pH of about 5.5; or at a pH of about 7.2; or at a pH of about 8.0. However, the medium can bind proteins at higher or lower pH.

Methods according to embodiments of the invention are not limited by the ionic strength of the fluid, and can be used with fluid having low and high ionic strengths. Many biological substances will readily adsorb to the medium at physiological ionic strength. Physiological ionic strength typically ranges from about 15 to about 20 mS/cm, although the ionic strength can be greater or lesser than those values. Typical salt concentrations that correspond to this range fall within about 0.1 to about 0.2 M, preferably 0.14 to about 0.17 M.

The temperature at which the fluid is placed in contacted with the medium varies between fluids and a given chromatographic material as is known in the art. Preferably, the temperature is ambient, but it can be higher or lower than ambient.

After the fluid has contacted the medium, the medium is preferably washed with an equilibration buffer as is known in the art. An equilibration buffer is a buffer that is preferably of the pH at which the fluid was contacted with the medium. Furthermore, the equilibration buffer washes from the medium substances that do not adsorb to the medium. Suitable equilibration buffers are known in the art, and include, for example, acetate buffer and phosphate buffered saline.

The desired component(s) typically adsorb(s) to the medium. In other embodiments, the desired component(s) may be removed in, for example, the equilibration buffer washing. In this case, the component(s) may be isolated from the buffer by methods known in the art.

Components that are adsorbed to the medium are subsequently desorbed in one embodiment by adjusting the pH to a value where the substance desorbs. The pH at which desorption occurs will depend upon the substance and upon a given medium. For example, for media that comprise an anion exchange moiety, desorption typically occurs over a pH gradient starting at about pH 8 and decreasing to about pH 3. For media that comprise a cation exchange moiety, the pH gradient applied typically starts at about pH 4 and is increased to about pH 11. For media that feature primarily hydrophobic groups, the pH gradient for desorption typically starts at about pH 7 and is decreased to about pH 3. For media that feature primarily hydrophobic groups, preferably an ionic strength gradient is also applied as described below. The pH can be adjusted by any routinely available reagent, such as aqueous solutions of Tris-HCl or carbonate buffers.

In some instances, as mentioned above, adjustment of the eluant ionic strength can increase effectiveness of the media. Thus, for media that comprise primarily hydrophobic groups, the ionic strength can be decreased concomitantly with pH. This is especially so for materials that additionally comprise —NH— moieties, which can give rise to mild ionic charges that become more effective as the ionic strength is decreased. The use of salt gradients is well-known in the art. Typically, salt concentrations for the present media need not exceed about 1.0 M, or about 0.5 M.

Typically, the desorbed desired component is subsequently collected, and can be further processed if desired. Typical purities of desired components, such as antibodies, that are purified in accordance with embodiments of the invention are about 70% or more, in some embodiments about 85% or more, and more preferably about 90% to about 99%.

Typically, chromatography cycles are repeated until the content of the source container is completely processed.

In those embodiments wherein a detector is not used, e.g., the method is carried out using a timer-controlled flow arrangement, a reference standard (e.g., a reference elution pattern, for example, establishing the compound of interest breakthrough profile from the chromatography device) is first determined, and the results are used to determine the number of chromatography cycles to process specific feed and time length for each of the operations in each of the cycles.

If desired, e.g., as part of cleaning and/or sanitation, fluid such as cleaning and/or sanitation buffer can be passed from one flow control arrangement to another, e.g., via a separate conduit communicating with the flow control arrangements, and the fluid can be passed to, for example, a waste container.

The following examples further illustrate the invention but, of course, should not be construed as in any way limiting its scope.

With the exceptions of references to the detector and time control arrangements, the system component identifiers are the same for Example 1 (referring to FIG. 1) and Example 3 (referring to FIG. 3), and are the same for Example 2 (referring to FIG. 2) and Example 4 (referring to FIG. 4).

### Example 1

This example demonstrates the use of a TFF device upstream of the chromatography column, and a product detector, in accordance with an embodiment of the invention.

A system is arranged as generally shown in FIG. 1. The chromatography sorbent within the column and the TFF device are equilibrated using the standard practice in the art before initiating the process operation.

A feed sample solution containing the desired component (target molecule) and impurities is pumped from the source container 2 (load tank) to the inlet of the TFF device 10 via lines 1 and 2. Optionally, feed sample solution can be passed from the source container into a mixing vessel 2A via line 1A, and from the mixing vessel to the inlet of the filtration device via lines 1B and 2.

The feed sample solution is passed through the inlet and tangentially across the surface of the porous filter element within the TFF device. Molecules smaller than the filter element pores pass through the element (i.e., providing the permeate) and molecules larger than the pores of the filter element are retained and enriched in fraction at the surface of the porous filter (i.e., providing the retentate). The target molecule and impurities larger than filter element pores are retained at the surface of the porous filter element pass through the second outlet (retentate outlet) of the filtration device. Impurities smaller than the filter element pores pass through the porous filter and pass through the first outlet (permeate outlet) of the TFF device along a second waste fluid flow path via line 4 to the waste tank 7.

The retentate from the filtration device, containing the target molecule and impurities larger than filter element pores, passes through the retentate outlet into the inlet of the chromatography column via lines 3, 5, and 6. Optionally, a surge tank can be used between the retentate outlet of the filtration device and the inlet of the chromatography column, such that the retentate passes via lines 3, 3A, 5, and 6.

The retentate, containing the target molecule and some impurities, passes into the chromatography column and contacts the sorbent, wherein some of the retentate including the target molecule binds to the sorbent and most of the other retentate (i.e., providing the effluent) including impurities and possibly some target, exits the chromatography column through the outlet and passes along the effluent fluid flow path via line 7 and along line 8 is directed along a first waste fluid flow path via line 10 to the waste tank 7.

When the product detector (sensor) positioned downstream of the outlet of the chromatography column detects the presence of the target molecule in the effluent from the chromatography column, this effluent is re-directed from the effluent fluid flow path along a desired component recycle fluid flow path via line 9 to the load tank where it is mixed with feed sample solution. Optionally, if the system also includes a mixing vessel, this effluent is re-directed from the effluent fluid flow path along a desired component recycle fluid flow path via line 9A to the mixing vessel where it is mixed with feed sample solution.

When the detector detects a level of the target molecule that equals or exceeds a pre-set value, corresponding to when the desired amount of target molecule is bound onto the chromatography sorbent (i.e., the desired amount is the binding capacity) within the chromatography column, the feed sample load is stopped on the chromatography column. After this is completed, a flush procedure is initiated on the chromatography column. Equilibration buffer is pumped from the equilibration buffer tank 3 via lines 12, 5, and 6 to the inlet of the chromatography column, wherein the buffer displaces unbound target molecule and impurities and passes them to the outlet of the chromatography column and along the desired component recycle flow path via line 9 to the load tank where it is mixed with the feed sample solution and directed to the inlet of the filtration device. If the system includes a mixing vessel, the displaced unbound target molecule and impurities are passed from the outlet of the chromatography column and along the desired component recycle flow path via line 9A to the mixing vessel where fluid is mixed with feed sample solution and directed to the inlet of the TFF device.

The flow path remains unchanged until the detector positioned downstream of the chromatography column fails to detect the target molecule or the detectable amount does not reach a preset value. The effluent from the outlet of the chromatography column is directed from the effluent fluid flow path along first waste fluid flow path to the waste tank. After this is completed, a wash procedure is initiated on the chromatography column. The wash buffer is pumped from the wash buffer tank 4 via lines 13, 5, and 6 to the inlet of the chromatography column. This releases some impurities that are weakly bound on the chromatography sorbent while the feed sample is applied on the chromatography column. The target molecule, which is captured during this period, remains bound. The effluent from the outlet of the chromatography column is still directed along the first waste fluid flow path to the waste tank during the wash procedure.

After a pre-defined amount of time, an elution procedure is initiated on the chromatography column. The elution buffer is pumped from the elution buffer tank 5 via lines 14, 5, and 6 to the inlet of the chromatography column. Simultaneously, flow from the outlet of the chromatography column is directed from the effluent fluid flow path along an eluted fluid flow path via line 11 to the product tank 8 (storage or elution tank) to collect the purified target molecule eluted from the chromatography sorbent.

When the detector positioned downstream of the chromatography column fails to detect the target molecule or the detectable amount does not reach a preset value, indicating the target molecule is completely eluted from the chromatography column, a cleaning procedure is initiated on the chromatography column. The cleaning solution is pumped from the cleaning solution tank 6 via lines 15, 5, and 6 to the inlet of the chromatography column. Simultaneously, flow from the outlet of the chromatography column is directed from the effluent fluid flow path along the first waste fluid flow path to the waste tank.

After a pre-defined amount of time, corresponding to the time required to completely remove any bound material from the chromatography column, a re-equilibration procedure is initiated on the chromatography column. The equilibration buffer is pumped from the equilibration buffer tank to the inlet of the chromatography column. Flow from the outlet of the chromatography column remains directed along the first waste fluid flow path to the waste tank during the re-equilibration procedure. Equilibration buffer is delivered to the chromatography column for a pre-defined amount of time. At this point a first process cycle is completed.

A second cycle is initiated by repeating the same procedure run for the first cycle. The process can be iteratively repeated for several cycles.

Toward the end of a process, as more and more liquid is removed from the system via the permeate outlet of the TFF device, the flow path from the permeate outlet of the filtration device (the second waste fluid flow path) is closed. Closing this flow path during the process halts the target molecule concentration process. At this point the volume of the system remains constant. The rest of the process is continued in order to continue capturing the target molecule on the chromatography column.

The process is ended when an acceptable amount of target molecule is captured and eluted from the chromatography column with minimal amount of target molecule still remaining in the sample load tank.

### Example 2

This example demonstrates the use of a TFF device downstream of the chromatography column, and a product detector, in accordance with an embodiment of the invention.

A system is arranged as generally shown in FIG. 2. The chromatography sorbent within the column and the TFF device are equilibrated using the standard practice in the art before initiating the process operation.

A feed sample solution containing the desired component (target molecule) and impurities is pumped from the source container 2 (load tank) via lines 101, 5, and 6 to the inlet of the chromatography column. Optionally, feed sample solution can be passed from the source container 2 into a mixing vessel 2A via line 101A, and from the mixing vessel to the inlet of the chromatography device via lines 101B, 5, and 6.

The feed sample solution, containing the target molecule and some impurities, is pumped through the inlet of the chromatography column and contacts the sorbent, wherein some of the feed sample solution including the target molecule binds to the sorbent and most of the other feed sample solution (i.e., the effluent) including impurities and possibly some target, exits the chromatography column through the outlet and passes along the effluent fluid flow path via lines 7 and 8 and is directed along the first waste fluid flow path via line 10 to the waste tank 7.

When the product detector positioned downstream of the outlet of the chromatography column detects the presence of the target molecule in the effluent from the chromatography column, this effluent is re-directed from the effluent fluid flow path along a desired component fluid flow path via lines 102 and 103 to the inlet of the TFF device 10. Optionally, a surge tank can be used between the outlet of the chromatography column and the inlet of the TFF device such that fluid passes along lines 102, 102A, and 103 to the inlet of the TFF device.

The re-directed effluent feed sample solution passes through the inlet and tangentially across the surface of the porous filter element within the TFF device. Molecules smaller than the filter element pores pass through the element (i.e., providing the permeate) and molecules larger than the pores of the filter element are retained and enriched in fraction at the surface of the porous filter (i.e., providing the retentate). The target molecule and impurities larger than filter element pores are retained at the surface of the porous filter element pass through the second outlet (retentate outlet) of the TFF device. Impurities smaller than the filter element pores pass through the porous filter and pass through the first outlet (permeate outlet) of the filtration device along a second waste fluid flow path via line 104 to the waste tank 7.

The flow from the retentate outlet is directed along the desired component recycle fluid flow path via line 109 to the load tank (or via line 109A to the mixing vessel) where it is mixed with feed sample solution and directed to the inlet of the chromatography column.

When the detector detects a level of the target molecule that equals or exceeds a pre-set value, corresponding to when the desired amount of target molecule is bound onto the chromatography sorbent (i.e., desired amount equals the binding capacity) within the chromatography column, the feed sample load is stopped on the chromatography column.

Equilibration buffer is pumped from the equilibration buffer tank 3 via lines 12, 5, and 6 to the inlet of the chromatography column, wherein the buffer displaces unbound target molecule and impurities and passes them to the outlet of the chromatography column and along the effluent fluid flow path via lines 7 and 8 and the flow is re-directed via lines 102 and 103 to the inlet of the TFF device.

The flow path remains unchanged until the detector positioned downstream of the chromatography column fails to detect the target molecule or the detectable amount does not reach a preset value. The effluent from the outlet of the chromatography column is directed from the effluent fluid flow path along the first waste fluid flow path via line 10 to the waste tank 7. After this is completed, a wash procedure is initiated on the chromatography column. The wash buffer is pumped from the wash buffer tank 4 via lines 13, 5, and 6 to the inlet of the chromatography column. This releases some impurities that are weakly bound on the chromatography sorbent while the feed sample is applied on the chromatography column. The target molecule, which is captured during this period, remains bound. The effluent from the outlet of the chromatography column is still directed from the effluent fluid flow path along the first waste fluid flow path to the waste tank during the wash procedure.

After a pre-defined amount of time, an elution procedure is initiated on the chromatography column. Elution buffer is pumped from the elution buffer tank 5 via lines 13, 5 and 6 to the inlet of the chromatography column. Simultaneously, flow from the outlet of the chromatography column is directed from the effluent fluid flow path along the eluted component fluid flow path via line 11 to the product tank 8 (elution or storage tank) to collect the purified target molecule eluted from the chromatography sorbent.

When the detector positioned downstream of the chromatography column fails to detect the target molecule or the detectable amount does not reach a preset value, indicating the target molecule is completely eluted from the chromatography column, a cleaning procedure is initiated on the chromatography column. Cleaning solution is pumped from the cleaning solution tank 6 via lines 15, 5 and 6 to the inlet of the chromatography column. Simultaneously, flow from the outlet of the chromatography column is directed from the effluent fluid flow path along the first waste fluid flow path to the waste tank.

After a pre-defined amount of time, corresponding to the time required to completely remove any bound material from the chromatography column, re-equilibration procedure is initiated on the chromatography column. Equilibration buffer is pumped from the equilibration buffer tank 3 via lines 12, 5, and 6 to the inlet of the chromatography column. Flow from the outlet of the chromatography column remains directed along the first waste fluid flow path to the waste tank during the re-equilibration procedure. The equilibration buffer is delivered to the chromatography column for a pre-defined amount of time. At this point a first process cycle is completed.

A second cycle is initiated by repeating the same procedure run for the first cycle. The process can be iteratively repeated for several cycles.

Toward the end of a process, as more and more liquid is removed from the system via the permeate outlet of the TFF device, the flow path from the permeate outlet of the filtration device (the second waste fluid flow path) is closed. Closing this flow path during the process halts the target molecule concentration process. At this point the volume of the system remains constant. The rest of the process is continued in order to continue capturing the target molecule on the chromatography column.

The process is ended when an acceptable amount of target molecule is captured and eluted from the chromatography column with minimal amount of target molecule still remaining in the sample load tank.

### Example 3

This example demonstrates the use of a TFF device upstream of the chromatography column, using a time controlled system (without a product detector) in accordance with an embodiment of the invention.

A system is arranged as generally shown in FIG. 3. The chromatography sorbent within the column and the TFF device are equilibrated using the standard practice in the art before initiating the process operation.

A series of parameters used during the process are deduced from previous reference experiments where feed sample solution containing the desired component (target molecule) and impurities is loaded at different flow rates and concentrations onto a laboratory scale chromatography column to obtain different product breakthrough profiles used to define (i) the VCF to apply on the TFF device to keep the concentration of the target molecule stable at the inlet of the chromatography column, (ii) the time at which the target molecule will break through the chromatography column and (iii) the sample load time to obtain the required binding capacity.

A feed sample solution containing the target molecule and impurities is pumped from the source container (load tank) to the inlet of the filtration device. The feed sample solution is passed through the inlet and tangentially across the surface of the porous filter element within the filtration device. Molecules smaller than the filter element pores pass through the element (i.e., providing the permeate) and molecules larger than the pores of the filter element are retained and enriched in fraction at the surface of the porous filter (i.e., providing the retentate). The target molecule and impurities larger than filter element pores are retained at the surface of the porous filter element pass through the second outlet (retentate outlet) of the filtration device. Impurities smaller than the filter element pores pass through the porous filter and pass through the first outlet (permeate outlet) of the filtration device along a second waste fluid flow path to the waste tank.

The TFF device is operated to set the VCF to keep the concentration of the desired (target) component stable at the second outlet (retentate outlet) of the TFF device and consequently at the inlet of the chromatography column. A flow rate is pre-defined, and the flow rate at the second outlet (retentate outlet) of the TFF device is set to match the pre-defined flow rate through the chromatography column. Flow rates at the inlet and permeate outlet are set to maintain the mass balance of target component in the chromatography system such that mass added via source container or mixing vessel equals the mass removed via the chromatography column.

The retentate from the TFF device, containing the target molecule and impurities larger than filter element pores, passes through the retentate outlet into the inlet of the chromatography column. Optionally, a surge tank can be used between the retentate outlet of the filtration device and the inlet of the chromatography column.

The retentate, containing the target molecule and some impurities, passes into the chromatography column and contacts the sorbent, wherein some of the retentate including the target molecule binds to the sorbent and most of the other retentate (i.e., providing the effluent) including impurities and possibly some target, exits the chromatography column through the outlet and passes along the effluent fluid flow path and is directed to the waste tank.

After a pre-defined amount of time, corresponding to the time for the target molecule to break through the chromatography column, the effluent from the chromatography column is directed from the effluent fluid flow path along the desired component recycle fluid flow path to the load tank (or to the mixing vessel) where it is mixed with feed sample solution and directed to the inlet of the filtration device.

After a pre-defined amount of time, corresponding to the time at which the desired amount of target molecule is bound onto the chromatography sorbent (i.e., desired amount equals binding capacity) within the chromatography column, the feed sample load on the chromatography column is stopped.

After this is completed, a flush procedure is initiated on the chromatography column. Equilibration buffer is pumped from the equilibration buffer tank to the inlet of the chromatography column, wherein the buffer displaces unbound target molecule and impurities and passes them to the outlet of the chromatography column and along the desired component recycle flow path to the load tank (or mixing vessel) where it is mixed with the feed sample solution and directed to the inlet of the filtration device. If the system includes a mixing vessel, the displaced unbound target molecule and impurities are passed from the outlet of the chromatography column and along the desired component recycle flow path to the mixing vessel where fluid is mixed with feed sample solution and directed to the inlet of the filtration device.

The flow path remains unchanged for a pre-defined amount of time (for example, the time required to pump a volume of equilibration buffer equivalent to 1.5 times the volume of the chromatography column). When that time is elapsed, the effluent from the outlet of the chromatography column is directed from the effluent fluid flow path along the first waste fluid flow path to the waste tank. After this is completed, a wash procedure is initiated on the chromatography column. The wash buffer is pumped from the wash buffer tank to the inlet of the chromatography column. This releases some impurities that are weakly bound on the chromatography sorbent while the feed sample is applied on the chromatography column. The target molecule, which is captured during this period, remains bound. The effluent from the outlet of the chromatography column is still directed from the effluent fluid flow path along the first waste fluid flow path to the waste tank during the wash procedure.

After a pre-defined amount of time, an elution procedure is initiated on the chromatography column. The elution buffer is pumped from the elution buffer tank to the inlet of the chromatography column. Simultaneously, the fluid flow from the outlet of the chromatography column is directed from the effluent fluid flow path along an eluted fluid flow path to the product tank to collect the purified target molecule eluted from the chromatography sorbent.

After a pre-defined amount of time, corresponding to the time required for the target molecule to be completely eluted from the chromatography column, a cleaning procedure is initiated on the chromatography column. The cleaning solution is pumped from the cleaning solution tank to the inlet of the chromatography column. Simultaneously, flow from the outlet of the chromatography column is directed from the effluent fluid flow path along the first waste fluid flow path to the waste tank.

After a pre-defined amount of time, corresponding to the time required to completely remove any bound material from the chromatography column, a re-equilibration procedure is initiated on the chromatography column. The equilibration buffer is pumped from the equilibration buffer tank to the inlet of the chromatography column. Flow from the outlet of the chromatography column remains directed along the first waste fluid flow path to the waste tank during the re-equilibration procedure. Equilibration buffer is delivered to the chromatography column for a pre-defined amount of time. At this point a first process cycle is completed.

A second cycle is initiated by repeating the same procedure run for the first cycle. The process can be iteratively repeated for several cycles.

Toward the end of a process, as more and more liquid is removed from the system via the permeate outlet of the filtration device, the flow path from the permeate outlet of the filtration device (the second waste fluid flow path) is closed. Closing this flow path during the process halts the target molecule concentration process. At this point the volume of the system remains constant. The rest of the process is continued in order to continue capturing the target molecule on the chromatography column.

The process is ended when an acceptable amount of target molecule is captured and eluted from the chromatography column with minimal amount of target molecule still remaining in the sample load tank.

### Example 4

This example demonstrates the use of a TFF device downstream of the chromatography column, using a time controlled system (without a product detector) in accordance with an embodiment of the invention.

A system is arranged as generally shown in FIG. 4. The chromatography sorbent within the column and the filtration device are equilibrated using the standard practice in the art before initiating the process operation.

Parameters are deduced to obtain product breakthrough profiles and define the (i) VCF to apply, (ii) the time at which the target molecule will breakthrough, and (iii) the sample load time to obtain the required binding capacity as described in Example 2.

A feed sample solution containing the target molecule and impurities is pumped from the source container (load tank) to the inlet of the chromatography column. Optionally, feed sample solution can be passed from the source container into a mixing vessel, and from the mixing vessel to the inlet of the chromatography device.

The feed sample solution, containing the target molecule and some impurities, is pumped through the inlet of the chromatography column and contacts the sorbent, wherein some of the feed sample solution including the target molecule binds to the sorbent and most of the other feed sample solution (i.e., the effluent) including impurities and possibly some target, exits the chromatography column through the outlet and passes along the effluent fluid flow path and is directed along the first waste fluid flow path to the waste tank.

After a pre-defined amount of time, corresponding to the time for the target molecule to break through the chromatography column, the effluent from the chromatography column is effluent is re-directed from the effluent fluid flow path along a desired component fluid flow path to the inlet of the filtration device. Optionally, a surge tank can be used between the outlet of the chromatography column and the inlet of the concentration unit.

The re-directed effluent feed sample solution passes through the inlet and tangentially across the surface of the porous filter element within the filtration device. Molecules smaller than the filter element pores pass through the element (i.e., providing the permeate) and molecules larger than the pores of the filter element are retained and enriched in fraction at the surface of the porous filter (i.e., providing the retentate). The target molecule and impurities larger than filter element pores are retained at the surface of the porous filter element pass through the second outlet (retentate outlet) of the filtration device. Impurities smaller than the filter element pores pass through the porous filter and pass through the first outlet (permeate outlet) of the filtration device along a second waste fluid flow path to the waste tank.

The flow from the retentate outlet is directed along the desired component recycle fluid flow path to the load tank (or to the mixing vessel) where it is mixed with feed sample solution and directed to the inlet of the chromatography column. If the system includes a mixing vessel, the flow from the retentate outlet is passed along the desired component recycle flow path to the mixing vessel where fluid is mixed with feed sample solution and directed to the inlet of the chromatography device.

The filtration device is operated to set the VCF to keep the concentration of the desired (target) component stable at the inlet of the chromatography column. The flow rate at the inlet of the TFF device is defined by the flow rate through the chromatography column. The flow rate at the permeate outlet is set to maintain the mass balance of target component in the chromatography system such that mass added via source container or mixing vessel equals the mass removed via the chromatography column. The flow rate at the retentate outlet is calculated from the flow rates at the inlet and outlet ports.

After a pre-defined amount of time, corresponding to the time at which the desired amount of target molecule is bound onto the chromatography sorbent (i.e., desired amount equals the binding capacity) within the chromatography column, the feed sample load is stopped on the chromatography column.

After this is completed, a flush procedure is initiated on the chromatography column. Equilibration buffer is pumped from the equilibration buffer tank to the inlet of the chromatography column, wherein the buffer displaces unbound target molecule and impurities and passes them to the outlet of the chromatography column and along the effluent fluid flow path the flow is re-directed to the inlet of the filtration device. The flow path remains unchanged for a pre-defined amount of time (for example, the time required to pump a volume of equilibration buffer equivalent to 1.5 times the volume of the chromatography column). When that time is elapsed, the effluent from the outlet of the chromatography column is directed from the effluent fluid flow path along the first waste fluid flow path to the waste tank.

After this is completed, a wash procedure is initiated on the chromatography column. The wash buffer is pumped from the wash buffer tank to the inlet of the chromatography column. This releases some impurities that are weakly bound on the chromatography sorbent while the feed sample is applied on the chromatography column. The target molecule, which is captured during this period, remains bound. The effluent from the outlet of the chromatography column is still directed from the effluent fluid flow path along the first waste fluid flow path to the waste tank during the wash procedure.

After a pre-defined amount of time, an elution procedure is initiated on the chromatography column. The elution buffer is pumped from the elution buffer tank to the inlet of the chromatography column. Simultaneously, the fluid flow from the outlet of the chromatography column is directed from the effluent fluid flow path along an eluted fluid flow path to the product tank to collect the purified target molecule eluted from the chromatography sorbent.

After a pre-defined amount of time, corresponding to the time required for the target molecule to be completely eluted from the chromatography column, a cleaning procedure is initiated on the chromatography column. The cleaning solution is pumped from the cleaning solution tank to the inlet of the chromatography column. Simultaneously, flow from the outlet of the chromatography column is directed from the effluent fluid flow path along the first waste fluid flow path to the waste tank.

After a pre-defined amount of time, corresponding to the time required to completely remove any bound material from the chromatography column, a re-equilibration procedure is initiated on the chromatography column. The equilibration buffer is pumped from the equilibration buffer tank to the inlet of the chromatography column. Flow from the outlet of the chromatography column remains directed along the first waste fluid flow path to the waste tank during the re-equilibration procedure. Equilibration buffer is delivered to the chromatography column for a pre-defined amount of time. At this point a first process cycle is completed.

A second cycle is initiated by repeating the same procedure run for the first cycle. The process can be iteratively repeated for several cycles.

Toward the end of a process, as more and more liquid is removed from the system via the permeate outlet of the filtration device, the flow path from the permeate outlet of the filtration device (the second waste fluid flow path) is closed. Closing this flow path during the process halts the target molecule concentration process. At this point the volume of the system remains constant. The rest of the process is continued in order to continue capturing the target molecule on the chromatography column.

The process is ended when an acceptable amount of target molecule is captured and eluted from the chromatography column with minimal amount of target molecule still remaining in the sample load tank.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A chromatography system comprising:
(a) a chromatography device for capturing at least one desired component, the device comprising a housing having an inlet and an outlet and providing a fluid flow path between the inlet and the outlet; and a chromatography medium disposed in the housing across the fluid flow path;
(b) a tangential flow filtration device, comprising a housing comprising an inlet, a first outlet, and a second outlet, defining a first fluid flow path between the inlet and the first outlet, and a second fluid flow path between the inlet and the second outlet, and a porous filter element comprising at least one porous membrane across the first fluid flow path, wherein the second outlet of the tangential flow filtration device is in fluid communication with the inlet of the chromatography device;
(c) an effluent fluid flow path in fluid communication with the outlet of the chromatography device, the effluent fluid flow path comprising at least a first conduit;
(d) (di) a sensor associated with the effluent fluid flow path, wherein the sensor detects the presence or absence of the desired component passing from the outlet of the chromatography device along the effluent fluid flow path, and provides a signal indicating of the presence or absence of the desired component, or
(dii) a time control arrangement, providing a signal indicating a set period of time with respect to the operation of the chromatograph device has elapsed;
(e) a first controllable flow control arrangement comprising a first valve, downstream of the outlet of the chromatography device, and downstream of the sensor if present, the first controllable flow control arrangement being in fluid communication with the effluent fluid flow path, wherein,
(ei) after the signal indicating of the presence or absence of the desired component is provided, or
(eii) after the signal indicating the set period of time has elapsed is provided;
the first flow control arrangement allows and/or prevents fluid flow along an eluted component fluid flow path, a first waste fluid flow path, and a desired component recycle fluid flow path; wherein the eluted component fluid flow path, the first waste fluid flow path, and the desired component recycle fluid flow path are downstream of, and in fluid communication with, the first flow control arrangement;
(f) the eluted component fluid flow path comprising at least a second conduit;
(g) an eluted component container, downstream of, and in fluid communication with, the eluted component fluid flow path;
(h) the first waste fluid flow path comprising at least a third conduit;
(i) a waste container, downstream of, and in fluid communication with, the first waste fluid flow path;
(j) the desired component recycle fluid flow path comprising at least a fourth conduit;
(k) a source container and/or a mixing vessel, suitable for containing a fluid comprising a desired component to be purified, the source container and/or the mixing vessel in fluid communication with both the desired component recycle fluid flow path and the inlet of the tangential flow filtration device.

2. A chromatography system comprising:
(a) a chromatography device for capturing at least one desired component, the device comprising a housing having an inlet and an outlet and providing a fluid flow path between the inlet and the outlet; and a chromatography medium disposed in the housing across the fluid flow path;
(b) an effluent fluid flow path in fluid communication with the outlet of the chromatography device, the effluent fluid flow path comprising at least a first conduit;
(c) (ci) a sensor associated with the effluent fluid flow path, wherein the sensor detects the presence or absence of the desired component passing from the outlet of the chromatography device along the effluent fluid flow path, and provides a signal indicating of the presence or absence of the desired component, or
(cii) a time control arrangement, providing a signal indicating a set period of time with respect to the operation of the chromatograpy device has elapsed;
(d) a first controllable flow control arrangement comprising a first valve, downstream of the outlet of the chromatography device, and downstream of the sensor if present, the first controllable flow control arrangement being in fluid communication with the effluent fluid flow path, wherein, after the signal indicative of the presence or absence of the desired component is provided, the flow control arrangement allows and/or prevents fluid flow along an eluted component fluid flow path, a first waste fluid flow path, and a desired component fluid flow path; wherein the eluted component fluid flow path, the first waste fluid flow path, and the desired component fluid flow path are downstream of, and in fluid communication with, the first flow control arrangement;
(e) the eluted component fluid flow path comprising at least a second conduit;
(f) an eluted component container, downstream of, and in fluid communication with, the eluted component fluid flow path;
(g) the first waste fluid flow path comprising at least a third conduit;
(h) a waste container, downstream of, and in fluid communication with, the first waste fluid flow path;
(i) the desired component fluid flow path comprising at least a fourth conduit;
(j) a tangential flow filtration device, comprising a housing comprising an inlet, a first outlet, and a second outlet, defining a first fluid flow path between the inlet and the first outlet, and a second fluid flow path between the inlet and the second outlet, and a porous filter element comprising at least one porous membrane across the first fluid flow path, wherein the inlet of the tangential flow filtration device is in fluid communication with the desired component fluid flow path, and the second outlet of the tangential flow filtration device is in fluid communication with a desired component recycle fluid flow path, the desired component recycle fluid flow path comprising at least a fifth conduit; and,
(k) a source container and/or a mixing vessel, suitable for containing a fluid comprising a desired component to be purified, the source container and/or the mixing vessel in fluid communication with both the second outlet of the tangential flow filtration device via the desired component recycle fluid flow path, and the inlet of the chromatography device.

3. The system of claim 1, further comprising a second waste fluid flow path, comprising an additional conduit, in fluid communication with the first outlet of the tangential flow filtration device and the waste container.

4. The system of claim 1, wherein the chromatography device comprises a chromatography column.

5. The system of claim 1, wherein the chromatography device comprises a chromatography monolith.

6. The system of claim 1, wherein the chromatography device comprises a chromatography capsule.

7. The system of claim 1, wherein the sensor detects the presence of the desired component compared to a pre-set value.

8. The system of claim 1, further comprising a second controllable flow control arrangement comprising a second valve, interposed between, and in fluid communication with, the second outlet of the tangential flow filtration device and the inlet of the chromatography device, wherein the additional controllable flow control arrangement allows and/or prevents fluid flow from the second outlet to the inlet of the chromatography device, and from one or more buffer containers to the inlet of the chromatography device.

9. The system of claim 2, further comprising a second controllable flow control arrangement comprising a second valve, interposed between, and in fluid communication with, the source container and/or the mixing vessel, and the inlet of the chromatography device, wherein the second controllable flow control arrangement allows and/or prevents fluid flow from the source container and/or the mixing vessel and from one or more buffer containers to the inlet of the chromatography device.

10. The system of claim 1, comprises a surge tank interposed between, and in fluid communication with, the second outlet of the tangential flow filtration device and the inlet of the chromatography device.

11. The system of claim 2, comprises a surge tank interposed between, and in fluid communication with, the effluent fluid flow path of the chromatography device, and the inlet of the tangential flow filtration device.

12. The system of claim 1, comprising the mixing vessel suitable for containing the fluid comprising a desired component to be purified, in fluid communication with both the desired component recycle fluid flow path and the inlet of the tangential flow filtration device, wherein the system further comprising a source container, also suitable containing the fluid comprising a desired component to be purified, upstream of, and in fluid communication with, the mixing vessel.

13. The system of claim 2, comprising the mixing vessel suitable for containing the fluid comprising a desired component to be purified, in fluid communication with both the second outlet of the tangential flow filtration device via the desired component fluid flow path, and the inlet of the chromatography device, wherein the system further comprising a source container, also suitable containing the fluid comprising a desired component to be purified, upstream of, and in fluid communication with, the mixing vessel.

14. A chromatographic method for obtaining at least one desired component, the method comprising:
(a) passing a fluid comprising at least one desired component from a source container or a mixing vessel and into a tangential flow filtration device, the tangential flow filtration device comprising a housing comprising an inlet, a first outlet, and a second outlet, defining a first fluid flow path between the inlet and the first outlet, and a second fluid flow path between the inlet and the second outlet, and a porous filter element having an upstream surface and a downstream surface and comprising a porous membrane across the first fluid flow path, wherein the second outlet of the tangential flow filtration device is in fluid communication with an inlet of a chromatography device; passing the fluid comprising at least one desired component tangentially to the upstream surface of the filter element; passing a desired component concentrated fluid tangentially to the upstream surface and along the second fluid flow path and through the second outlet into the inlet of the chromatography device; and passing a desired component reduced fluid along the first fluid flow path through the filter element and through the first outlet along a second waste fluid flow path to a waste container;
(b) passing the desired component concentrated fluid into the chromatography device, the device comprising a housing having the inlet and an outlet and providing a fluid flow path between the inlet and the outlet; and a chromatography medium for binding the at least one desired component, disposed in the housing across the fluid flow path;
(c) passing fluid from the outlet of the chromatography device along an effluent fluid flow path, including
(ci) detecting the presence or absence of the desired component(s) in the fluid, or
(cii) determining whether one or more set periods of time have elapsed, the set periods of time comprising a first waste fluid flow time, a desired component recycle fluid flow time, and a eluted component fluid flow path time;
(d) opening and/or closing one or more of the following flow paths: a desired component recycle fluid flow path, a first waste fluid flow path, and an eluted component fluid flow path, wherein,
(di) if the desired component(s) is not detected, or detected at less than a pre-set value, or
(dii) if the desired component recycle fluid flow time has not elapsed,
the desired component recycle fluid flow path is opened, and the first waste fluid flow path and the eluted component fluid flow path are closed; and,
(diii) if the desired component(s) is detected at equal to, or greater than, the pre-set value, or
(div) if the desired component recycle fluid flow time has not elapsed,
the desired component recycle fluid flow path is opened or remains open, and the first waste fluid flow path and the eluted component fluid flow path are closed or remain closed; and,
(dv) if after the desired component(s) have previously been detected at equal to, or greater than, the pre-set value, and the desired component(s) is subsequently not detected, or detected at less than the pre-set value, or
(dvi) if the desired component recycle fluid flow time has elapsed,
the desired component recycle fluid flow path and the eluted component fluid flow path are closed, and the waste fluid flow path is opened;
(e) passing fluid along the opened fluid flow path, wherein, if the first waste fluid flow path is opened, the method includes passing fluid along the first waste fluid flow path into a waste container, and if the desired component recycle fluid flow path is opened, the method includes passing fluid along the desired component recycle fluid flow path into the source container or the mixing vessel, and passing fluid from the source container or from the mixing vessel into the tangential fluid flow filter device; and
(f) repeating (a), (b), (c), (d) and (e) at least once.

15. A chromatographic method for obtaining at least one desired component, the method comprising:
(a) passing a fluid comprising at least one desired component from a source container or a mixing vessel and into an inlet of a chromatography device, and through the chromatography device, the device comprising a housing having the inlet and an outlet and providing a fluid flow path between the inlet and the outlet; and a chromatography medium for binding the at least one desired component, disposed in the housing across the fluid flow path;
(b) passing fluid from the outlet of the chromatography device along an effluent fluid flow path, including
(bi) detecting the presence or absence of the desired component(s) in the fluid, or
(bii) determining whether one or more set periods of time have elapsed, the set periods of time comprising a first waste fluid flow time, a desired component fluid flow time, and an eluted component fluid flow path time;
(c) opening and/or closing one or more of the following flow paths: a desired component fluid flow path, a first waste fluid flow path, and an eluted component fluid flow path, wherein,
(ci) if the desired component(s) is not detected, or detected at less than a pre-set value, or
(cii) if the desired component fluid flow time has not elapsed, the desired component fluid flow path is opened, and the first waste fluid flow path and the eluted component fluid flow path are closed; and,
(ciii) if the desired component(s) is detected at equal to, or greater than, the pre-set value, or
(civ) if the desired component fluid flow time has not elapsed, the desired component fluid flow path is opened or remains open, and the first waste fluid flow path and the eluted component fluid flow path are closed or remain closed; and,
(cv) if after the desired component(s) have previously been detected at equal to, or greater than, the pre-set value, and the desired component(s) is subsequently not detected, or detected at less than the pre-set value, or
(cvi) if the desired component fluid flow time has elapsed,
the desired component fluid flow path and the eluted component fluid flow path are closed, and the waste fluid flow path is opened;
(d) passing fluid along the opened fluid flow path, wherein, if the first waste fluid flow path is opened, the method includes passing fluid along the first waste fluid flow path into a waste container, and if the desired component fluid flow path is opened, the method includes passing fluid along the desired component fluid flow path into an inlet of a tangential fluid flow filter device, the tangential flow filtration device comprising a housing comprising the inlet, a first outlet, and a second outlet, defining a first fluid flow path between the inlet and the first outlet, and a second fluid flow path between the inlet and the second outlet, and a porous filter element having an upstream surface and a downstream surface and comprising a porous membrane across the first fluid flow path, wherein the second outlet of the tangential flow filtration device is in fluid communication with the source container or the mixing vessel via a desired component recycle fluid flow path, wherein the source container or the mixing vessel is in fluid communication with both the second outlet of the tangential flow filtration device, and the inlet of the chromatography device;
(e) passing the fluid comprising at least one desired component tangentially to the upstream surface of the filter element; passing a desired component concentrated fluid tangentially to the upstream surface and along the second fluid flow path and through the second outlet along the desired component recycle fluid flow path into the source container or the mixing vessel; and passing a desired component reduced fluid along the first fluid flow path through the filter element and through the first outlet along a second waste fluid flow path to the waste container; and
(f) repeating (a), (b), (c), (d) and (e) at least once.

16. The method of claim 14, further comprising:
(g) closing the desired component recycle fluid flow path or the desired component fluid flow path, and the first waste fluid flow path, and opening the eluted component fluid flow path; and;
(h) passing an elution fluid through the chromatography device, and passing desired component(s) containing elution fluid from the outlet of the chromatograph device along the eluted component fluid flow path into the eluted component container.

17. The system of claim 2, wherein the sensor detects the presence of the desired component compared to a pre-set value.

18. The system of claim 2, further comprising a second waste fluid flow path, comprising an additional conduit, in fluid communication with the first outlet of the tangential flow filtration device and the waste container.

19. The system of claim 2, wherein the chromatography device comprises a chromatography column.

20. The method of claim 15, further comprising:
(g) closing the desired component recycle fluid flow path or the desired component fluid flow path, and the first waste fluid flow path, and opening the eluted component fluid flow path; and;
(h) passing an elution fluid through the chromatography device, and passing desired component(s) containing elution fluid from the outlet of the chromatograph device along the eluted component fluid flow path into the eluted component container.
